(19) United States
(12) Patent Application Publication
(10) Pub. No.: US 2007/0185145 A1
(43) Pub. Date: Aug. 9, 2007

(54) Pharmaceutical composition containing a central opioid agonist, a central opioid antagonist, and a peripheral opioid antagonist, and method for making the same

(76) Inventor: Robert B. Royds, Plainsboro, NJ (US)

Correspondence Address: WATOV & KIPNES, P.C., P.O. Box 247, Princeton Junction, NJ, 08550, US

(21) Appl. No.: 11/346,657

(22) Filed: Feb. 3, 2006

(51) Int. Cl. A61K 31/485

(52) U.S. Cl. 514/282

(57) ABSTRACT

A pharmaceutical composition for treating or preventing a disease, condition or symptoms thereof in a warm-blooded animal including a human, includes a therapeutically effective amount of an opioid agonist exhibiting potential pharmacologically addictive properties in warm blooded animals including humans; a side-effect reducing agent present in amounts sufficient to at least substantially neutralize the adverse side effects of the opioid agonist; an opioid antagonist present in a sequestered form in amounts sufficient to block the pharmacological effect of the opioid agonist upon release from the sequestered form; and a pharmaceutically acceptable carrier.

### FIELD OF THE INVENTION

The present invention relates to pharmaceutical compositions, and more particularly to a pharmaceutical composition containing an opioid agonist yielding a tamper resistant dosage formulation with enhanced safety profile, and reduced abuse potential.

### BACKGROUND OF THE INVENTION

Opiods or opioid agonists are a class of drugs that are known to be effective analgesics in the moderate to strong range. Despite extensive research to date, no analgesics have been found that are more effective for severe pain. This class of drugs include morphine, the archetypical opioid, and various others such as, for example, codeine, dihydrocodeine, hydrocodone, hydromorphone, levorphanol, meperidine, buprenorphine, fentanyl, fentanyl derivatives, dipipanone, heroin, tramadol, etorphine, dihydroetorphine, butorphanol, methadone, diamorphine, oxycodone, oxymorphone, pethidine and propoxyphene.

Opioid agonists chemically interact with areas or binding sites of the central nervous system related to the perception of pain, to movement, mood and behavior, and to the regulation of neuroendocrinological functions. Opioid agonists exhibit pharmacological properties that provide a range of therapeutic uses for patients in addition to analgesic use. Opioid agonists have been prescribed for effective use as hypnotics, sedatives, anti-diarrheals, anti-spasmodics, and anti-tussives. Unfortunately, the use of opioid agonists has also been associated with a number of undesirable side effects in patients, and, due to the addictive properties, has been subjected to illegal diversion for abuse by addicts.

One of the advantages of opioid agonists is that there is no upper limit to the dosage and the achievable pain relief as long as the dose is increased gradually to develop tolerance against adverse effects particularly respiratory depression. Some adverse side effects include constipation, respiratory depression, nausea, vomiting, dizziness, orthostatic hypotension, drowsiness, urinary retention, itch, dry mouth, headache, miosis, changes in mood and mental clouding resulting without resulting loss of consciousness. Both therapeutic and chronic use of opioid agonists has further been observed to compromise the function of the immune system. Opioid agonists decrease the proliferation of macrophage progenitor cells and lymphocytes, and affect cell differentiation. Opioid agonists can also inhibit leukocyte migration. Such adverse side effects typically limit the dosage a patient can tolerate, and therefore can limit their effective use.

One common adverse side effect experienced by patients is constipation, which can undesirably progress to bowel obstruction, fecal impaction, or paralytic ileus. Opioid agonist-induced changes in gastrointestinal motility are almost universal when used to treat pain, and at times can be a primary factor for limiting their use, leaving the patient in pain. Because these conditions can require physical or surgical intervention if left unchanged, stimulant laxatives are generally given as an adjunct to prevent these complications. Physiological tolerance does not develop with regard to constipation. It should be noted that in some therapeutic regimens (such as those aimed at treating diarrhea), mild constipation would be a desired effect and hence laxatives would not be given.

The possibility of developing psychological dependence (i.e., addiction) is another major concern in the use of the treatment of pain with centrally-acting opioid agonists. Opioid agonists produce central nervous system effects in the body, which generate changes in mood, levels of awareness or perceptions, and sensations. Such effects are also known to produce varying degrees of compulsive drug use in people. Many opioid agonists produce euphoria when ingested orally, intravenously, subcutaneously, through the nasal membranes, or when smoked. This euphoria causes dependence. Tolerance to euphoria develops rapidly; a regular user can require orders of magnitude more drug than a naive user. The ability of opioid agonists to block pain, both physical and emotional, is linked to another cause of dependence. The diversion of these drugs from the patient in pain to another for an illicit purpose, e.g., to an addict, has been problematic.

Increased abuse of pharmaceutical opioid agonists has recently been reported especially with the advent of extended release opioid agonist dosage forms. Extended release opioid agonist dosage forms are formulated for decreased dosing frequency. Therefore, each dosage form contains an amount of opioid agonist, which would otherwise be administered in several immediate release dosages. Therefore, such extended release dosage forms contain substantially increased amounts of opioid agonist per dose. A single extended release dosage form such as a tablet can provide much more opioid agonist to the potential abuser than a low dose, immediate release dosage form. This results in a stronger feeling of euphoria, or “high” from extended release dosage forms than an abuser would get from an immediate release one. This makes such dosage forms more desirable for an abuser to illicitly divert.

Previous attempts at abuse resistant opioid agonist compositions for oral administration have been made to curb such diversions. One includes incorporating one or more opioid antagonists such as, for example, naloxone or naltrexone, which rapidly reverses the effects of the opioid agonist. These competitive antagonists are drugs that bind to the corresponding opioid receptors principally in the central nervous system with higher affinity than agonists, but do not activate the receptors. As a result, the interaction of the antagonist with the receptors displaces the agonists, and attenuates and/or reverses the central agonist effects. Some therapeutic formulations include a mix of agonist and antagonists to mediate the effects in order to reduce the abuse and dependence liability as compared with full opioid agonists.

Further attempts have included formulating an opioid agonist which has substantial activity orally as well as activity when administered by injection, in combination with an opioid antagonist which is less effective orally than by injection. The combination helps prevent abuse involving crushing and dissolving the composition followed by injection. Most prescription opioid analgesic pharmaceutical compositions are tablets designed for oral administration. Therefore opioid antagonists, which have very low oral bioavailability, have little action when taken orally at parenterally effective doses. Therefore, the opioid antagonist has little effect when the tablet is taken as intended but greatly enhanced effect if the tablet is abused parenterally.

Such opioid antagonists have substantially increased effect when taken directly into the blood stream. Thus, abusing the opioid by crushing the tablet, dissolving it, and injecting or snorting (intranasal administration), would cause the antagonist to have its full effect, essentially blocking the central nervous system opioid receptors, preventing the abuser from receiving an opioid agonist effect, and inducing unpleasant withdrawal symptoms almost immediately in opioid-dependent individuals.

Another attempt at discouraging abuse includes formulating a dosage form having an opioid agonist in a releasable form, and a sequestered opioid antagonist that is substantially not released when the dosage form is administered intact. The ratio of the amount of opioid antagonist released from the dosage form after tampering to the amount of the agonist released from the intact dosage form is 4:1 or greater. However, while this can help deter abuse involving the crushing of a tablet, there is still a need for abuse resistant opioid agonist pharmaceutical compositions that also counteracts or at least reduces the adverse side effects often associated with the use of opioid agonists in the patient for greater safety and efficacy. The present invention is directed to such a pharmaceutical composition.

Accordingly, there is a need for a pharmaceutical composition containing an opioid agonist that offers safe and effective treatment of pain and other ailments with less adverse side effects from the opioid agonist in warm-blooded animals including humans, while substantially reducing the potential for abuse of the opioid agonist contained therein. There is a need for a pharmaceutical composition containing an opioid agonist that is resistant to misuse, abuse or diversion without diminishing the therapeutic effects of the opioid agonist or incurring the risk of precipitating withdrawal symptoms and conditions thereof, when administered intact.

### SUMMARY OF THE INVENTION

The present invention relates generally to a pharmaceutical composition containing an opioid agonist for treating or preventing a disease, condition or symptoms thereof in a warm-blooded animal including a human, and a method for reducing adverse side effects and reducing the potential for abuse of an opioid agonist. The pharmaceutical composition of the present invention is formulated to relieve the peripherally mediated adverse side effects typically associated with the opioid agonist for greater flexibility in dosing and administration. The pharmaceutical composition of the present invention is further formulated to prevent illicit diversion of the opioid agonist for non-medical or non-therapeutic use. This combination of reduced adverse side effects and illicit diversion prevention yields a pharmaceutical composition with an enhanced safety profile and therapeutic effectiveness, while at least maintaining or preserving the therapeutic analgesic efficacy of the opioid agonist.

The pharmaceutical composition of the present invention is generally in the form of a solid dosage formulation comprising a therapeutic amount of an opioid agonist, a neutralizing opioid antagonist in a sequestered form for preventing diversion, and a side-effect reducing agent corresponding to the opioid agonist. The opioid agonist provides a therapeutic effect for the relief of pain and other ailments including disease, conditions and symptoms thereof. The neutralizing opioid antagonist reverses or neutralizes the pharmacological effect of the opioid agonist when released from the composition and into the bloodstream to deter abuse and diversion. The side-effect reducing agent produces a specific pharmacological effect to at least reduce or counteract the peripheral adverse side effects of the corresponding opioid agonist.

The pharmaceutical composition of the present invention preferably comprises an opioid agonist, an opioid antagonist in a sequestered form, and at least one side-effect reducing agent for substantially reducing or counteracting the adverse side effects of the opioid agonist. Preferably, the pharmaceutical composition of the present invention is in the form of a solid dosage formulation wherein the opioid antagonist is present in a substantially non-releasable form (i.e., sequestered). More preferably, the solid dosage formulation is oral.

In preferred embodiments, the pharmaceutical composition comprises a therapeutically effective amount of the opioid agonist in combination with at least one side-effect reducing agent, to provide a corresponding desirable therapeutic effect (e.g., analgesic) with substantial reduction or relief from adverse side-effects typically associated with the opioid agonist for greater dosing flexibility and use. Since the opioid antagonist is present in a substantially non-releasable form, the sequestered opioid antagonist does not substantially block the therapeutic effect (e.g., analgesic) of the opioid agonist, and does not pose a risk of precipitation of withdrawal in opioid tolerant or dependent patients, when the pharmaceutical composition is orally administered intact.

In one aspect of the present invention, there is provided a pharmaceutical composition for treating or preventing a disease, condition or symptoms thereof in a warm-blooded animal including a human, comprising:

a therapeutically effective amount of an opioid agonist exhibiting potential pharmacologically addictive properties in warm blooded animals including humans;

a side-effect reducing agent present in amounts sufficient to at least substantially neutralize the adverse side effects of the opioid agonist;

an opioid antagonist present in a sequestered form in amounts sufficient to block the pharmacological effect of the opioid agonist upon release from the sequestered form; and

a pharmaceutically acceptable carrier.

In another aspect of the present invention, there is provided a method for reducing the adverse side effects and potential for abuse of an opioid agonist in a warm-blooded animal including a human, the method comprising preparing the pharmaceutical composition comprising a therapeutically effective amount of an opioid agonist exhibiting potential pharmacologically addictive properties in warm blooded animals including humans; a side-effect reducing agent present in amounts sufficient to at least substantially neutralize the peripheral adverse side effects of the opioid agonist; an opioid antagonist present in a sequestered form in amounts sufficient to block the pharmacological effect of the opioid agonist upon release from the sequestered form; and a pharmaceutically acceptable carrier.

In a further aspect of the present invention, there is provided a method for treating or preventing a disease, condition or symptoms thereof in a warm-blooded animal including a human, comprising administrating to the warm-blooded animal suffering from the disease, condition or symptoms thereof a therapeutically effective amount of the pharmaceutical composition disclosed herein.

### DETAILED DESCRIPTION OF THE INVENTION

The present invention is directed to a pharmaceutical composition for treating or preventing a disease, condition or symptoms thereof, and a method for substantially reducing or counteracting adverse side effects and minimizing the potential for abuse of an opioid agonist in a warm-blooded animal including a human. The present invention provides a mechanism or method for preparing a combination dosage form in which the opioid agonist and a corresponding side effect-reducing agent are each released from the dosage form, and an opioid antagonist for preventing abuse is retained in a sequestered form. This combination allows the agonist and the side-effect reducing agent to be delivered to the patient, in a manner to provide a therapeutic effect together with substantial reduction or neutralization of adverse side effects typically associated with the opioid agonist and/or increased opioid potency is provided throughout the dosing period. The pharmaceutical composition of the present invention is preferably in the form of a solid dosage form comprising an opioid agonist in combination with an opioid antagonist in a sequestered form, and a side effect-reducing agent present in amounts sufficient to at least substantially reduce or counteract the adverse side effects of the opioid agonist.

In preferred embodiments, the dosage formulation comprises a therapeutically effective amount of the opioid agonist in combination with at least one side-effect reducing agent, to provide a corresponding desirable therapeutic effect (e.g., analgesic) with minimal adverse side-effects for greater dosing flexibility and use. Since the opioid antagonist is present in a substantially non-releasable form, the sequestered opioid antagonist does not substantially block the therapeutic effect (e.g., analgesic) of the opioid agonist when the dosage formulation is orally administered intact, and therefore does not pose a risk of precipitation of withdrawal in opioid tolerant or dependent patients. The side effect-reducing agent effectively enhances the therapeutic potency of the opioid agonist, while easing the adverse side effects of the opioid agonist, including, but not limited to, constipation, respiratory depression, nausea, vomiting, dizziness, orthostatic hypotension, drowsiness, urinary retention, itch, dry mouth, headache, miosis, changes in mood and mental clouding resulting without resulting loss of consciousness.

The term “opioid agonist” is defined for purposes of the present invention to mean any opioid-based compound including opioid peptides, opium alkaloids, semi-synthetic and fully synthetic opioids, capable of binding to an opioid receptor and triggering a response in a cell, and include bimodally acting opioid agonists. The term “opioid agonist” can be used interchangeably with the term “opioid.”

Suitable examples of opioid agonists useful in the present invention, include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, combinations thereof, salts thereof, and the like.

Preferred examples include hydrocodone, morphine, hydromorphone, oxycodone, codeine, levorphanol, meperidine, methadone, salts thereof, and combinations thereof.

The term “side effect-reducing agent” is defined for purposes of the present invention to mean any opioid-based or non-opioid-based pharmacologically active compound capable of substantially reducing or countering the adverse side effects typically associated with opioid agonists in warm-blooded animals including humans. Preferred side effect-reducing agents are selected from those known to reduce or counteract adverse side effects such as constipation, respiratory depression, nausea, vomiting, dizziness, orthostatic hypotension, drowsiness, urinary retention, itch, dry mouth, headache, miosis, negative changes in mood, mental clouding, reduction of macrophage progenitor cells and lymphocytes, inhibition of leukocyte migration, and the like. More preferred side effect-reducing agents are those that are known to reduce or counteract constipation, respiratory depression, nausea, vomiting, dizziness, and urinary retention.

Suitable examples of a side effect-reducing agent include cathartics such as methylnaltrexone, bisoxatin acetate, casanthranol, danthron, docusate calcium, docusate sodium, emodin, frangulin, glucofrangulin, lactulose, magnesium carbonate hydroxide, magnesium chloride, magnesium citrate, magnesium hydroxide, magnesium lactate, magnesium phosphate, dibasic, magnesium sulfate, mercurous chloride, mercury mass, oxyphenistan acetate, phenolphthalein, phenolphthalol, phenoltetrachlorophthalein, picosulfate sodium, poloxamers, potassium bisulfate, potassium bitartrate, potassium phosphate, dibasic, potassium sodium tartrate, potassium sulfate, potassium sulfite, potassium tartrate, prostaglandins, senna, sennoside, sodium phosphate, dibasic, sodium succinate, sodium tartrate, sulsatin, triacetyldiphenolisatin, yellow phenolphthalein, anti-emetics such as 5-Hydroxytryptamine antagonists including, but not limited to, Dolasetron, Granisetron, Ondansetron, and Tropisetron, Dopamine antagonists including, but not limited to, Domperidone, Droperidol, Haloperidol, Chlorpromazine and metoclopramide, antihistamines (i.e., 5HT2 receptor antagonists) including, but not limited to, Cyclizine, Diphenhydramine, Dimenhydrinate, and cannabinoids including but not limited to, Marinol, respiratory stimulants such as almitrine, bemegride, cropropamide, crotethamide, dimefline, dimorpholamine, doxapram, ethamivan, fominoben, lobeline, mepixanox, metamivam, nikethamide, picrotoxin, pimeclone, pyridofylline, sodium succinate, tacrine; and immune system enhancing agents to augment the number and proliferation of T-cells and NK cell activity such as Shitake mushroom powder, and the like.

The term “opioid antagonist” is defined for purposes of the present invention to mean any opioid-based compound capable of binding to the same opioid receptor of a corresponding opioid agonist, and preventing or blocking the activation of the receptor.

The combination of opioid agonist and side effect-reducing agent can be formulated as a controlled-release oral dosage form, including tablets and capsules. In preferred embodiments, the controlled-release oral dosage form provides a controlled release of an opioid agonist and a controlled-release of a side effect-reducing agent, such that when the dosage form is administered to a warm-blooded animal including a human, the blood levels of the agonist is maintained throughout the dosing period at a therapeutically effective level, and the side effect-reducing agent at a level sufficient to decrease the side effects associated with the opioid agonist but not sufficient to negate the therapeutic effect of the opioid agonist.

In preferred embodiments of the present invention, the ratio of the opioid agonist to the side effect-reducing agent in the controlled-release oral dosage form is from about 1:1 to about 100:1 by weight based on the total weight of the composition. In preferred embodiments, the ratio of the opioid agonist with the side effect-reducing agent is about 4:1 to about 50:1 by weight, more preferably about 20:1. In other preferred embodiments of the invention the amount of the side effect-reducing agent administered is about 10 to about 1000 fold less than the amount of the opioid agonist administered.

The pharmaceutical composition of the present invention can be formulated for as oral dosage forms and sublingual dosage forms.

The pharmaceutical compositions comprising the combination of the present invention can conveniently be presented in unit dosage forms and can be prepared by any of the methods well known in the art of pharmacy. Such methods generally include the step of bringing the combination into association with a carrier which constitutes one or more accessory ingredients. The pharmaceutical compositions of the present invention suitable for oral administration can be presented as discrete units such as capsules, cachets, tablets, or lozenges, each containing a predetermined amount of the active ingredient.

A tablet can be made by compression or molding, optionally with one or more accessory ingredients. Compressed tablets can be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier can be made by molding in a suitable machine.

In addition to the aforementioned ingredients, the pharmaceutical composition of the present invention can further include one or more accessory ingredient(s) selected from excipients, fillers, adjuvants, coating agents, capsulating agents, buffers, flavoring agents, binders, disintegrants, surface active agents, thickeners, lubricants, preservatives (including antioxidants), and the like.

Examples of solid compositions for oral administration include tablets, troches, sublingual tablets, capsules, pills, granules and the like. The solid composition can be prepared by mixing one or more active ingredients with at least one inactive diluents. The composition can further contain additives other than the inactive diluents, for example, a lubricant, a disintegrator and a stabilizer. Tablets and pills can be coated with an enteric or gastroenteric film, if necessary. They can be covered with two or more layers. They can also be adsorbed to a sustained release material, or microcapsulated. Additionally, the compositions can be capsulated by means of an easily degradable material such gelatin. They can be further dissolved in an appropriate solvent such as fatty acid or its mono-, di- or triglyceride to be a soft capsule. Sublingual tablet can be used.

In the present invention, a dosage form can include one active ingredient only or a combination of two or more active ingredients. When a combination of a plurality of active ingredients are used, their respective contents can be suitably increased or decreased in consideration of their effects and safety.

The composition of, the present invention can further include other pharmacologically active ingredients as far as they do not contradict the purpose of the present invention.

Controlled-release oral dosage forms according to the present invention can be prepared using the methods available to one skilled in the art. In certain embodiments of the present invention, controlled-release tablets comprise the opioid agonist and side effect-reducing agent in a controlled release matrix. The controlled-release matrix can include hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the opioid can be used in accordance with the present invention. The opioid agonist particles can, alternatively or additionally, be film coated with a material that permits release of the opioid agonist at a sustained rate in an aqueous medium. The film coat is chosen so as to achieve, in combination with the other stated properties, a desired in-vivo release rate. The sustained release coating formulations of the present invention should be capable of producing a strong, continuous film that is smooth and elegant, capable of supporting pigments and other coating additives, non-toxic, inert, and tack-free.

The dosage forms comprising an opioid agonist and side effect-reducing agent can optionally be coated with one or more materials suitable for the regulation of the opioid agonist release or for the protection of the formulation. In one embodiment of the present invention, coatings are provided to permit either pH-dependent or pH-independent release, e.g., when exposed to gastrointestinal fluid.

A pH-dependent coating serves to release the opioid in desired areas of the gastrointestinal (GI) tract, e.g., the stomach or small intestine, such that an absorption profile is provided which is capable of providing at least about eight hours and preferably about twelve hours to up to about twenty-four hours of therapeutic benefit to a patient. When a pH-independent coating is desired, the coating is designed to achieve optimal release of the opioid regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings to obtain formulations can also impart a repeat-action effect whereby unprotected drug is coated over the enteric coat and is released in the stomach, while the remainder, being protected by the enteric coating, is released further down the gastrointestinal tract. Coatings which are pH-dependent can be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

The pharmaceutical composition of the present invention is further formulated to liberate the opioid antagonist under conditions of misuse or tampering. The opioid antagonist is present in the present pharmaceutical composition in a sequestered or substantially non-releasable form. In one embodiment of the present invention, the opioid antagonist is present in a substantially non-releasable form that comprises opioid antagonist particles coated with a coating that substantially prevents its release. In a preferred embodiment of the present invention, such coating surrounding and encapsulating the antagonist particles is impermeable to the drug and is insoluble in the oral cavity, mucosal areas and gastrointestinal system.

When the pharmaceutical composition of the present invention is orally administered to a warm-blooded animal including a human, the opioid antagonist is not substantially released from the coating and is, therefore, not available for absorption into the body. Thus, the opioid antagonist, although present in the pharmaceutical composition, does not substantially block the therapeutic effectiveness of the opioid agonist. However, if the pharmaceutical composition has been tampered with as to compromise the integrity of the coating, the opioid antagonist contained therein is made available to at least partially block the effect of the opioid agonist. In this manner, the opioid antagonist acts to suppress the “euphoric” effect, and precipitously produce unpleasant withdrawal symptoms in opioid dependant users. This feature, thus, decreases the potential for abuse or diversion of the opioid in the pharmaceutical composition.

In this manner, if a user attempts to abuse the opioid agonist contained in the pharmaceutical composition of the present invention in the form of an oral solid dosage formulation by, e.g., chewing, grinding, crushing, or dissolving it in an organic solvent, which can be in the presence of heat (e.g., greater than about 45° C.), the coating will be damaged and will no longer prevent the opioid antagonist from being released. Upon administration, the opioid antagonist will be released and significantly block the euphoric effect of the opioid agonist.

In one preferred embodiment of the present invention, the pharmaceutical composition further includes a portion (e.g., tablet core bead, matrix particle) containing the opioid antagonist is coated with a hydrophobic material selected from (i) an alkylcellulose; (ii) an acrylic polymer; or (iii) mixtures thereof. The coating can be applied in the form of an organic or aqueous solution or dispersion. The coating can be applied to obtain a weight gain from about 2 to about 25% of the substrate in order to obtain a desired sustained release profile as known in the art.

The ratio of the opiod agonist to the coated opioid antagonist is such that when the oral dosage form is tampered with as to compromise the integrity of the coating that renders the opioid antagonist substantially releasable, the euphoric of the opioid agonist is negated by the opioid antagonist when misused by a human subject orally, pareterally, intranasally, or sublingually. Preferably, the euphoric effect of the opioid agonist is negated by the opioid antagonist when misused parenterally or sublingually.

The opioid antagonist in a substantially non-releasable form comprises an opioid antagonist dispersed in a matrix that renders the antagonist sequestered or substantially non-releasable, wherein the matrix comprises one or more of a pharmaceutically acceptable hydrophobic material. The antagonist is substantially not released from the matrix, thus is not made available to be absorbed during its transit through the gastrointestinal system.

In preferred embodiments, the opioid antagonist of the present invention includes naltrexone, nalmefene, cyclazacine, levallorphan and mixtures thereof. In more preferred embodiments, the opioid antagonist is naloxone or naltrexone. In certain embodiments, the amount of the opioid antagonist, present in a substantially non-releasable form, can be about 10 mg to 275 mg.

In certain embodiments of the present invention, the ratio of the opioid agonist to the substantially non-releasable form of an opioid antagonist in the oral dosage form is such that the effect of the opioid agonist is at least partially blocked when the dosage form is chewed, crushed or dissolved in a solvent and heated, and administered orally, intranasally, parenterally or sublingually. Since the oral dosage form of the present invention, when administered properly as intended, does not substantially release the opioid antagonist, the amount of such antagonist can be varied more widely than if the opioid antagonist is available to be released into the gastrointestinal system upon oral administration. For safety reasons, the amount of the antagonist present in a substantially non-releasable form must not be harmful to humans even if fully released. The ratio of particular opioid agonist to antagonist can be determined without undue experimentation by one skilled in the art.

In certain embodiments of the present invention, the ratio of the opioid agonist and the opioid antagonist, present in a substantially non-releasable form, is about 1:1 to about 50:1 by weight, preferably about 1:1 to about 20:1 by weight. In certain preferred embodiments, the ratio is about 1:1 to about 10:1 by weight. In a preferred embodiment of the invention, the opioid agonist comprises oxycodone or hydrocodone and is present in the amount of about 15 mg to 45 mg and the opioid antagonist comprises naltrexone and is present in about 0.5 mg to 5 mg.

In one preferred embodiment of the present invention, an opioid antagonist in a substantially non-releasable form can be prepared by combining the antagonist with one or more of a pharmaceutically acceptable hydrophobic material. For example, opioid antagonist particles can be coated with a coating that substantially prevents the release of the antagonist, the coating comprising the hydrophobic materials(s). Another example is an opioid antagonist that is dispersed in a matrix that renders the antagonist to be substantially non-releasable, the matrix comprising the hydrophobic materials(s).

In another embodiment of the present invention, the pharmaceutical acceptable hydrophobic material comprises a cellulose polymer selected from the group consisting of ethylcellulose, cellulose acetate, cellulose propionate (lower, medium or higher molecular weight), cellulose acetate propionate, cellulose acetate butyrate, cellulose acetate phthalate and cellulose triacetate. An example of ethylcellulose is one that has an ethoxy content of from about 44% to 55%. Ethylcellulose can be used in the form of an alcoholic solution. In certain other embodiments, the hydrophobic material comprises polylactic acid, polyglycolic acid or a co-polymer of the polylactic and polyglycolic acid.

In certain embodiments of the present invention, the hydrophobic material can comprises a cellulose polymer selected from the group consisting of cellulose ether, cellulose ester, cellulose ester ether, and cellulose. Representative materials include a polymer selected from the group consisting of cellulose acylate, cellulose diacylate, cellulose triacylate, cellulose acetate, cellulose diacetate, cellulose triacetate, mono, di, and tricellulose alkanylates, mono-, di-, and tri-cellulose aroylates, and mono, di, and tricellulose alkenylates.

Additional cellulose polymers useful for preparing an opioid antagonist in a substantially non-releasable form includes acetaldehyde dimethyl cellulose acetate, cellulose acetate ethylcarbamate, cellulose acetate methylcarbamate, and cellulose acetate dimethylaminocellulose acetate.

An acrylic polymer useful for preparation of the opioid antagonist in a substantially non-releasable form includes, but are not limited to, acrylic resins comprising copolymers synthesized from acrylic and methacrylic acid esters (e.g., the copolymer of acrylic acid lower alkyl ester and methacrylic acid lower alkyl ester) containing about 0.02 to 0.03 mole of a tri (lower alkyl) ammonium group per mole of the acrylic and methacrylic monomers used. An example of a suitable acrylic resin is a polymer manufactured by Rohm Pharma GmbH and sold under the Eudragit® RS trademark. Eudragit RS30D is preferred. Eudragit® RS is a water insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins such as Eudragit® RS can be used in the form of an aqueous suspension.

In certain embodiments of the invention, the acrylic polymer can be selected from the group consisting of acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate co-polymers.

When the opioid antagonist in a substantially non-releasable form comprises opioid antagonist particles coated with a coating that renders the antagonist substantially non-releasable, and when a cellulose polymer or an acrylic polymer is used for preparation of the coating composition, suitable plasticizers, e.g., acetyl triethyl citrate and/or acetyl tributyl citrate can also be admixed with the polymer. The coating can also contain additives such as coloring agents, talc and/or magnesium stearate, which are well known in the coating art.

The coating composition can be applied onto the opioid antagonist particles by spraying it onto the particles using any suitable spray equipment known in the part. For example, a Wuster fluidized-bed system can be used in which an air jet, injected from underneath, fluidizes the coated material and effects drying while the insoluble polymer coating is sprayed on. The thickness of the coating will depend on the characteristics of the particular coating composition being used. However, it is well within the ability of one skilled in the art to determine by routine experimentation the optimum thickness of a particular coating required for a particular dosage form of the present invention.

The pharmaceutically acceptable hydrophobic material useful for preparing an opioid antagonist in a substantially non-releasable form includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesthers, polydioxanone, polygluconate, polylactic-acid-polyethylene oxide copolymers, poly(hydroxybutyrate), polyphosphoesther or mixtures or blends of any of these.

In certain embodiments, the biodegradable polymer comprises a poly(lactic/glycolic acid), a copolymer of lactic and glycolic acid, having molecular weight of about 2,000 to about 500,000 daltons. The ratio of lactic acid to glycolic acid is from about 100:0 to about 25:75, with the ratio of lactic acid to glycolic acid of 65:35 being preferred.

Once the opioid antagonist in a substantially non-releasable form is prepared, it is combined with the opioid agonist and the side effect-reducing agent of the present invention, along with conventional excipients known in the art, to prepare the oral dosage form of the present invention.

In certain preferred embodiments of the invention, the oral dosage form is a capsule or a tablet. When formulated as a tablet, the opioid antagonist and agonist can be combined with one or more inert, non-toxic pharmaceutical excipients which are suitable for the manufacture of tablets. Such excipients include, for example, an inert diluent such as lactose; granulating and disintegrating agents such as cornstarch; binding agents such as starch; and lubricating agents such as magnesium stearate.

The oral dosage form of the present invention can be formulated to provide immediate release of the opioid agonist and side effect-reducing agent contained therein. In other embodiments of the invention, however, the oral dosage form provides sustained-release of the opioid agonist and side effect-reducing agent.

In certain embodiments, the oral dosage forms providing sustained release of the opioid agonist can be prepared by admixing the opioid antagonist in a substantially non-releasable form with the agonist and the side effect-reducing agent, and desirable pharmaceutical excipients to provide a tablet, and then coating the tablet with a sustained-release tablet coating.

In certain embodiments of the invention, sustained release opioid agonist tablets can be prepared by admixing the substantially non-releasable form of an opioid antagonist with an opioid antagonist and a side effect-reducing agent in a matrix that provides the tablets with sustained-releasing properties.

A combination of the opioid agonist, the side effect-reducing agent and a substantially non-releasable form of an opioid antagonist can be formulated as a controlled or sustained release oral formulation in any suitable tablet, coated tablet or multiparticulate formulation known to those skilled in the art. The sustained release dosage form can optionally include a sustained release carrier which is incorporated into a matrix along with the opioid agonist and the side effect-reducing agent, and a non-available form of an opioid antagonist, or can be applied as a sustained release coating.

In embodiments in which the opioid agonist comprises hydrocodone, the sustained release oral dosage forms can include analgesic doses from about 8 mg to about 50 mg of hydrocodone per dosage unit. In sustained release oral dosage forms where hydromorphone is the therapeutically active opioid, it is included in an amount from about 2 mg to about 64 mg hydromorphone hydrochloride. In another embodiment, the opioid agonist comprises morphine, and the sustained release oral dosage forms of the present invention include from about 2.5 mg to about 800 mg morphine by weight. In yet another embodiment, the opioid agonist comprises oxycodone and the sustained release oral dosage forms include from about 2.5 mg to about 800 mg oxycodone. In certain preferred embodiments, the sustained release oral dosage forms include from about 20 mg to about 30 mg oxycodone. Controlled release oxycodone formulations are known in the art. The opioid agonist can comprise tramadol and the sustained release oral dosage forms can include from about 25 mg to 800 mg tramadol per dosage unit. The dosage form can contain more than one opioid agonist to provide a substantially equivalent therapeutic effect. Alternatively, the dosage form can contain molar equivalent amounts of other salts of the opioid agonists useful in the present invention.

In one preferred embodiment of the present invention, the sustained release dosage form comprises such particles comprising the opioid agonist and side effect-reducing agent, wherein the particles have diameter from about 0.1 mm to about 2.5 mm, preferably from about 0.5 mm to about 2 mm.

The opioid agonist particles and side effect-reducing agent particles are preferably film coated with a material that permits release of the opioid agonist at a sustained rate in an aqueous medium. The film coat is chosen so as to achieve, in combination with the other stated properties, a desired in-vivo release rate. The sustained release coating formulations of the present invention produces a strong, continuous film that is smooth and elegant, capable of supporting pigments and other coating additives, non-toxic, inert, and tack-free.

The dosage forms comprising an opioid agonist, a side effect-reducing agent and a substantially non-releasable opioid antagonist can optionally be coated with one or more materials suitable for the regulation of the opioid agonist release or for the protection of the formulation. In one embodiment, coatings are provided to permit either pH-dependent or pH-independent release, e.g., when exposed to gastrointestinal fluid. A pH-dependent coating serves to release the opioid in desired areas of the gastro-intestinal (GI) tract, e.g., the stomach or small intestine, such that an absorption profile is provided which is capable of providing at least about eight hours and preferably about twelve hours to up to about twenty-four hours of analgesia to a patient. When a pH-independent coating is desired, the coating is designed to achieve optimal release of the opioid regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

In another embodiment of the present invention, there is provided a method for reducing the potential for abuse and adverse side effects of an opioid agonist in a warm-blooded animal including a human, the method comprising preparing the pharmaceutical composition comprising a therapeutically effective amount of an opioid agonist exhibiting potential pharmacologically addictive properties in warm blooded animals including humans; a side-effect reducing agent present in amounts sufficient to at least substantially neutralize the adverse side effects of the opioid agonist; an opioid antagonist present in a sequestered form in amounts sufficient to block the pharmacological effect of the opioid agonist upon release from the sequestered form; and a pharmaceutically acceptable carrier.

In a further embodiment of the present invention, there is provided a method for treating a disease, condition or symptoms thereof in a warm-blooded animal including a human, comprising administrating a pharmaceutical composition as disclosed herein, in a pain relieving effective amount to the warm-blooded animal.

The term “treatment” or “treating” used heroin includes any means of control such as prevention, care, relief of the condition, attenuation of the condition and arrest of progression.

In one application of the present invention, the present pharmaceutical composition has been found to be effective in the treatment and prevention of pain in warm-blooded animals, including humans. The opioid agonist provides an analgesic effect for the relief of pain. The present pharmaceutical composition can also be formulated for various uses such as, for example, hypnotics, sedatives, anti-diarrheals, anti-spasmodics, and anti-tussives.

In such a combination of the opioid agonist, and the side effect-reducing agent, the dosage of the opioid agonist for providing therapeutic benefits, and the dosage of the side effect-reducing agent can be independently determined for incorporation into a unitary composition.

Subjects to be treated by the methods of the present invention include warm-blooded animals, preferably mammalian animals and more preferably humans. Depending on the specific condition being treated, the subjects can be administered the opioid agonist and side effect-reducing agent, in combination with the sequestered opioid antagonist, at any suitable therapeutically effective amount as determined by one having skill in the art.

In general, the therapeutically effective amount of the opioid agonist for therapeutic use can be widely varied in the broad practice of the present invention, depending on the age, gender, body weight of patient, the specific application or desired therapeutic effect, disease, condition, and symptoms thereof involved, administration route, term of treatment, and the like, as readily determinable within the skill of the art. The mode of administration and dosage forms will affect the therapeutically effective amounts of the opioid agonist which are desirable and efficacious for a given treatment regimen. Suitable therapeutically effective amounts of the opioid agonist for achievement of therapeutic benefit in treatment can be in the range of from about 1 μg to 150 mg per kilogram body weight of the patient per day, preferably in the range of 10 μg to 100 mg per kilogram body weight of the patient per day and more preferably 50 μg to 75 mg per kilogram body weight of the patient per day. The therapeutically effective amount is preferably presented two, three, four, five, six, or more doses administered at appropriate intervals throughout the day. These doses can be administered in unit dosage forms, for example containing 10 μg to 1000 mg, preferably from 50 μg to 500 mg, and more preferably 50 μg to 250 mg active ingredient per unit dosage form.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

What is claimed is:

1. A pharmaceutical composition for treating or preventing a disease, condition or symptoms thereof in a warm-blooded animal including a human, comprising:
a therapeutically effective amount of an opioid agonist exhibiting potential pharmacologically addictive properties in warm blooded animals including humans;
a side-effect reducing agent in amounts sufficient to at least substantially neutralize the adverse side effects of the opioid agonist
an opioid antagonist present in a sequestered form in amounts sufficient to block the pharmacological effect of the opioid agonist upon release from the sequestered form; and
a pharmaceutically acceptable carrier.

2. The pharmaceutical composition of claim 1, wherein the opioid agonist is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, combinations thereof, and salts thereof.

3. The pharmaceutical composition of claim 1, wherein the opioid agonist is selected from the group consisting of hydrocodone, morphine, hydromorphone, oxycodone, codeine, levorphanol, meperidine, methadone, salts thereof, and combinations thereof.

4. The pharmaceutical composition of claim 1, wherein the therapeutically effective amount of the opioid agonist is from about 1 μg to 150 mg per kilogram body weight of the warm-blooded animal.

5. The pharmaceutical composition of claim 1, wherein the therapeutically effective amount of the opioid agonist is from about 10 μg to 100 mg per kilogram body weight of the warm-blooded animal.

6. The pharmaceutical composition of claim 1, wherein the therapeutically effective amount of the opioid agonist is from about 50 μg to 75 mg per kilogram body weight of the warm-blooded animal.

7. The pharmaceutical composition of claim 1, wherein the therapeutically effective amount of the opioid agonist is from about 10 μg to 1000 mg.

8. The pharmaceutical composition of claim 1, wherein the therapeutically effective amount of the opioid agonist is from about 50 μg to 500 mg.

9. The pharmaceutical composition of claim 1, wherein the therapeutically effective amount of the opioid agonist is from about 50 μg to 250 mg.

10. The pharmaceutical composition of claim 1, wherein it is in the form of a solid dosage form.

11. The pharmaceutical composition of claim 10, wherein the solid dosage form is selected from a group consisting of a tablet, a capsule, a cachet, a lozenge, a troche, a sublingual tablet, a pill, and a granule.

12. The pharmaceutical composition of claim 1, wherein the side effect-reducing agent is selected from the group consisting of a peripheral opiate antagonist, a cathartic, an anti-emetic, a respiratory stimulant, and immune system enhancers.

13. The pharmaceutical composition of claim 1, wherein the side effect-reducing agent is selected from the group consisting of bisoxatin acetate, casanthranol, danthron, docusate calcium, docusate sodium, emodin, frangulin, glucofrangulin, lactulose, magnesium carbonate hydroxide, magnesium chloride, magnesium citrate, magnesium hydroxide, magnesium lactate, magnesium phosphate, dibasic, magnesium sulfate, mercurous chloride, mercury mass, oxyphenistan acetate, phenolphthalein, phenolphthalol, phenoltetrachlorophthalein, picosulfate sodium, poloxamers, potassium bisulfate, potassium bitartrate, potassium phosphate, dibasic, potassium sodium tartrate, potassium sulfate, potassium sulfite, potassium tartrate, prostaglandins, senna, sennoside, sodium phosphate, dibasic, sodium succinate, sodium tartrate, sulsatin, triacetyldiphenolisatin, yellow phenolphthalein, 5-Hydroxytryptamine antagonists, dopamine antagonists, 5HT2 receptor antagonists, cannabinoids, almitrine, bemegride, cropropamide, crotethamide, dimefline, dimorpholamine, doxapram, ethamivan, fominoben, lobeline, mepixanox, metamivam, nikethamide, picrotoxin, pimeclone, pyridofylline, sodium succinate, tacrine, and combinations thereof.

14. The pharmaceutical composition of claim 1, wherein the side effect-reducing agent is present in a ratio amount of side effect reducing agent to opioid agonist ranging from about 1:1 to 1:100.

15. The pharmaceutical composition of claim 1, wherein the side effect-reducing agent is present in a ratio amount of side effect reducing agent to opioid agonist ranging from about 1:40 to 1:50.

16. The pharmaceutical composition of claim 1, wherein the side effect-reducing agent is present in a ratio amount of side effect reducing agent to opioid agonist at about 1:20.

17. The pharmaceutical composition of claim 1, wherein the opioid antagonist is selected from the group consisting of naltrexone, nalmefene, cyclazacine, levallorphan and combinations thereof.

18. The pharmaceutical composition of claim 1, wherein the opioid antagonist is present in a ratio amount of opioid antagonist to agonist ranging from about 1:1 to 1:50.

19. The pharmaceutical composition of claim 1, wherein the opioid antagonist is present in a ratio amount of opioid antagonist to agonist ranging from about 1:1 to 1:20.

20. The pharmaceutical composition of claim 1, wherein the opioid antagonist is present in a ratio amount of opioid antagonist to agonist ranging from about 1:1 to 1:10.

21. The pharmaceutical composition of claim 12, wherein said peripheral opiate antagonist includes methylnaltrexone.

22. A method for reducing the adverse side effects and potential for abuse of an opioid agonist in a warm-blooded animal including a human, the method comprising preparing the pharmaceutical composition of claim 1.

23. A method for treating or preventing a disease, condition or symptoms thereof in a warm-blooded animal including a human, comprising administrating to the warm-blooded animal suffering from the disease, condition or symptoms thereof a therapeutically effective amount of the pharmaceutical composition of claim 1.
